SEQUENCE LISTING (24 sequences)

SEQ ID NO: 1 - protein, 11 aa, synthetic construct
RASENIYSNLA

SEQ ID NO: 2 - protein, 7 aa, synthetic construct
AATNLQS

SEQ ID NO: 3 - protein, 7 aa, synthetic construct
AATNLQD

SEQ ID NO: 4 - protein, 7 aa, synthetic construct
AATSLQS

SEQ ID NO: 5 - protein, 9 aa, synthetic construct
QHLWGTPYT

SEQ ID NO: 6 - protein, 10 aa, synthetic construct
GFSLTSYGLS

SEQ ID NO: 7 - protein, 17 aa, synthetic construct
YIWYDGNTNFHPSLKSR

SEQ ID NO: 8 - protein, 17 aa, synthetic construct
YIWYDGNTNFHSSLKSR

SEQ ID NO: 9 - protein, 17 aa, synthetic construct
YIWYDGNTNFHSPLKSR

SEQ ID NO: 10 - protein, 17 aa, synthetic construct
TEGHYYGSNYGYYALDY

SEQ ID NO: 11 - protein, 107 aa, synthetic construct
DIQMTQSPSSLSASVGDRVTITCRASENIYSNLAWYQQKPGKAPKLLLYAATNLQSGVPSRFSGSGSGTDFTLTISSLQPEDFATYYCQHLWGTPYTFGGGTKLEIK

SEQ ID NO: 12 - protein, 107 aa, synthetic construct
DIQMTQSPSSLSASVGDRVTITCRASENIYSNLAWYQQKQGKAPKLLLYAATNLQDGVPSRFSGSGSGTDYTLTISSLQPEDFATYFCQHLWGTPYTFGQGTKLEIK

SEQ ID NO: 13 - protein, 107 aa, synthetic construct
DIQMTQSPSSLSASVGDRVTITCRASENIYSNLAWYQQKPGKAPKLLIYAATSLQSGVPSRFSGSGSGTDFTLTISSLQPEDFATYYCQHLWGTPYTFGGGTKVEIK

SEQ ID NO: 14 - protein, 125 aa, synthetic construct
QVQLQESGPGLVKPSETLSLTCTVSGFSLTSYGLSWIRQPPGKGLEWIGYIWYDGNTNFHPSLKSRVTISKDTSKNQFSLKLSSVTAADTAVYYCAKTEGHYYGSNYGYYALDYWGQGTSVTVSS

SEQ ID NO: 15 - protein, 125 aa, synthetic construct
QVQLQESGPGLVKPSETLSLTCTVSGFSLTSYGLSWIRQPPGKGLEWIGYIWYDGNTNFHSSLKSRVTISKDTSKSQVSLKLSSVTAADTAVYYCAKTEGHYYGSNYGYYALDYWGQGTLVTVSS

SEQ ID NO: 16 - protein, 125 aa, synthetic construct
QVQLQESGPGLVKPSETLSLTCTVSGFSLTSYGLSWIRQPPGKGLEWIGYIWYDGNTNFHSPLKSRVTISVDTSKNQFSLKLSSVTAADTAVYYCAKTEGHYYGSNYGYYALDYWGQGTLVTVSS

SEQ ID NO: 17 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 454 aa, synthetic construct
QVQLQESGPGLVKPSETLSLTCTVSGFSLTSYGLSWIRQPPGKGLEWIGYIWYDGNTNFHPSLKSRVTISKDTSKNQFSLKLSSVTAADTAVYYCAKTEGHYYGSNYGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 454 aa, synthetic construct
QVQLQESGPGLVKPSETLSLTCTVSGFSLTSYGLSWIRQPPGKGLEWIGYIWYDGNTNFHSSLKSRVTISKDTSKSQVSLKLSSVTAADTAVYYCAKTEGHYYGSNYGYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 454 aa, synthetic construct
QVQLQESGPGLVKPSETLSLTCTVSGFSLTSYGLSWIRQPPGKGLEWIGYIWYDGNTNFHSPLKSRVTISVDTSKNQFSLKLSSVTAADTAVYYCAKTEGHYYGSNYGYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 214 aa, synthetic construct
DIQMTQSPSSLSASVGDRVTITCRASENIYSNLAWYQQKPGKAPKLLLYAATNLQSGVPSRFSGSGSGTDFTLTISSLQPEDFATYYCQHLWGTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 214 aa, unidentified
DIQMTQSPSSLSASVGDRVTITCRASENIYSNLAWYQQKQGKAPKLLLYAATNLQDGVPSRFSGSGSGTDYTLTISSLQPEDFATYFCQHLWGTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, synthetic construct
DIQMTQSPSSLSASVGDRVTITCRASENIYSNLAWYQQKPGKAPKLLIYAATSLQSGVPSRFSGSGSGTDFTLTISSLQPEDFATYYCQHLWGTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0092112 A1
(43) Pub. Date: Apr. 2, 2026

(54) USE OF ANTI-CTLA-4 ANTIBODIES FOR TREATING ADENOID CYSTIC CARCINOMA

(71) Applicant: OncoC4, Inc., Rockville, MD (US)

(72) Inventors: Yang Liu, Potomac, MD (US); Pan Zheng, Potomac, MD (US)

(21) Appl. No.: 19/112,015

(22) PCT Filed: Sep. 15, 2023
(86) PCT No.: PCT/US2023/074364

§ 371 Date: Mar. 14, 2025

Related U.S. Application Data
(60) Provisional application No. 63/376,043, filed on Sep. 16, 2022.

(51) Int. Cl. C07K 16/28; A61K 9/00; A61K 38/00; A61P 35/04

(52) U.S. Cl. CPC C07K 16/2818; A61K 9/0019; A61K 38/00; A61P 35/04; C07K 2317/51; C07K 2317/565

(57) ABSTRACT

The present invention relates to uses of anti-CTLA-4 antibodies for treating adenoid cystic carcinoma.

### FEDERALLY SPONSORED RESEARCH OR DEVELOPMENT

This invention was made in part with Government support under Grant Number R44CA250824 awarded by National Cancer Institute, NIH. The Government has certain rights in this invention.

### FIELD OF THE INVENTION

The invention relates to the use of anti-CTLA-4 antibodies for treating adenoid cystic carcinoma (ACC), including as a sole immunotherapy or as a monotherapy.

### REFERENCE TO THE SEQUENCE LISTING

Applicant hereby refers to the Sequence Listing that is contained in the file “111005_0604_01PC00_Sequence_Listing.xml” (25 kB; created on Sep. 14, 2023), the contents of which are incorporated herein by reference.

### BACKGROUND OF THE INVENTION

ACC is a rare cancer with an annual incidence of approximately 4 per million and 16-year limited duration prevalence of approximately 33 per million. In the U.S., there are about 1400 new cases per year and about 11,000 patients live with ACC. ACC usually originates from major and minor salivary glands, but can also arise from mammary, prostate, lacrimal, and other secretary gland tissues. The latter are managed with the same principles as salivary primaries. ACC is typically initially treated with curative-intent surgery and/or radiation, but most of the time it relapses. Approximately 50% of these cases become metastatic, often in the lungs. Almost all recurrent and/or metastatic (R/M) ACC is fatal, because there are no curative therapies currently available and only a limited number of effective treatments. No therapies have been FDA approved for use in ACC patients. Outside of clinical trials, chemotherapy or multi-targeted tyrosine kinase inhibitors are used with palliative intent in R/M ACC patients based on limited phase II data demonstrating modest clinical efficacy. Accordingly, there is a need in the art for an effective treatment for ACC.

### SUMMARY OF THE INVENTION

Provided herein is a method of treating an adenoid cystic carcinoma (ACC) in a subject in need thereof, which may comprise administering an anti-CTLA-4 antibody to the subject. Also provided herein provided are an anti-CTLA-4 antibody for use in treating an ACC, a composition comprising an anti-CTLA-4 antibody for treating an ACC, and use of an anti-CTLA-4 antibody in the manufacture of a medicament for treating an ACC. The ACC may be recurrent, metastatic, or both. The anti-CTLA-4 antibody may be the only immunotherapeutic agent administered to the subject or which is intended for use in treating the ACC. The anti-CTLA-4 antibody may be administered to the subject or intended for use as a monotherapy.

The anti-CTLA-4 antibody may be Ipilimumab/Yervoy, XTX101, Botensilimab, Zalifrelimab, ADG116, HBM4003, APL-509, BA3071, BMS-986249, TikAb, quavonlimab (MK-1308), or a combination thereof. The anti-CTLA-4 antibody may comprise (a) a light chain variable region comprising a complementarity determining region (CDR) 1 comprising the amino acid sequence set forth in SEQ ID NO: 1; a CDR2 comprising the amino acid sequence set forth in any one of SEQ ID NOs: 2-4; and, a CDR3 comprising the amino acid sequence set forth in SEQ ID NO: 5; and, (b) a heavy chain variable region comprising comprising a CDR1 comprising the amino acid sequence set forth in SEQ ID NO: 6; a CDR2 comprising the amino acid sequence set forth in any one of SEQ ID NOs: 7-9; and, a CDR3 comprising the amino acid sequence set forth in SEQ ID NO: 10. The anti-CTLA-4 antibody may comprise a light chain variable region comprising a CDR2 comprising the sequence set forth in SEQ ID NO: 3 and heavy chain variable region comprising a CDR2 comprising the sequence set forth in SEQ ID NO: 9. The anti-CTLA-4 antibody may comprise a light chain variable region comprising the sequence set forth in SEQ ID NO: 12 and a heavy chain variable region comprising the sequence set forth in SEQ ID NO: 16. The anti-CTLA-4 antibody may comprise a light chain comprising the sequence set forth in SEQ ID NO: 23, and a heavy chain comprising the sequence set forth in SEQ ID NO: 21. The anti-CTLA-4 antibody may be ONC-392. The anti-CTLA-4 antibody may have been diluted with 5% Dextrose Solution to a final concentration of about 1.0 to about 2.5 mg/mL. The dilution may have been from a formulation containing 5 mg/mL anti-CTLA-4 antibody, 20 mM histidine buffer, 8.8% (w/v) trehalose dihydrate, and 0.06% (w/v) PS80 at pH 6.0.

Also provided herein is a composition comprising an anti-CTLA-4 antibody. The antibody may comprise a light chain variable region comprising a CDR1 comprising the amino acid sequence set forth in SEQ ID NO: 1; a CDR2 comprising the amino acid sequence set forth in SEQ ID NO: 3; and, a CDR3 comprising the amino acid sequence set forth in SEQ ID NO: 5. The antibody may further comprise a heavy chain variable region comprising a CDR1 comprising the amino acid sequence set forth in SEQ ID NO: 6; a CDR2 comprising the amino acid sequence set forth in SEQ ID NO: 9; and, a CDR3 comprising the amino acid sequence set forth in SEQ ID NO: 10. The antibody may have been diluted with 5% Dextrose Solution to a final concentration of about 1.0 to about 2.5 mg/mL. The dilution may have been from a formulation containing 5 mg/mL anti-CTLA-4 antibody, 20 mM histidine buffer, 8.8% (w/v) trehalose dihydrate, and 0.06% (w/v) PS80 at pH 6.0. The antibody may comprise a light chain variable region comprising the sequence set forth in SEQ ID NO: 12 and a heavy chain variable region comprising the sequence set forth in SEQ ID NO: 16. The antibody may comprise a light chain comprising the sequence set forth in SEQ ID NO: 23 and a heavy chain comprising the sequence set forth in SEQ ID NO: 21.

### BRIEF DESCRIPTION OF THE DRAWINGS

FIG. 1 shows a diagram of Part A of an ONC-392 monotherapy clinical trial.

FIG. 2 shows a diagram of Part B of a phase I clinical trial of ONC-392 plus Pembrolizumab.

FIG. 3 shows a diagram of Part B of the phase IB expansion of a trial of ONC-392 plus Pembrolizumab.

FIG. 4A-B show the study design and preliminary outcomes of a clinical trial of ONC-392. FIG. 4A shows the dose finding study design. FIG. 4B shows the preliminary outcomes. * unconfirmed as of Oct. 13, 2021. #Surgical tissue IHC demonstrated heavy CD4+ and CD8+ T cell infiltration within the tumor.

FIG. 5 shows the design of a clinical trial to test the efficacy of anti-CTLA-4 antibody ONC-392 for treating relapsed/metastatic (R/M) ACC.

FIGS. 6A-C show magnetic resonance imaging (MRI) images demonstrating the efficacy of anti-CTLA-4 antibody ONC-392 against ACC. FIG. 6A shows the target lesion at baseline (Dec. 9, 2021), which was an anterior left temporal soft tissue tumor mass. FIG. 6B shows the target lesion after two cycles of treatment with ONC-392 (on Feb. 24, 2022). FIG. 6C shows the target lesion after 3 cycles of ONC-392 treatment (on Mar. 24, 2022).

### DETAILED DESCRIPTION

The anti-CTLA-4 antibodies described herein can be used to treat cancer. In particular, the inventors have discovered that, surprisingly, anti-CTLA-4 antibodies—even provided as a sole immunotherapeutic agent—are effective against adenoid cystic carcinoma, which is a rare cancer that is difficult to treat. No anti-CTLA-4 monoclonal antibody has been shown to effectively treat ACC when provided as a sole immunotherapeutic agent. ACC may be considered as cold tumors due to its low mutation/neoantigen burden. But, the prevalent MYB-NFIB gene fusion can lead to neoantigen for T cells, and intratumor infiltrates were observed in 42% of clinical samples of both primary and metastatic lesions. Interestingly, none of the tumors analyzed had PD-L.1 expression on tumor cells. Compared to tumors that do not have immune infiltrates, transcripts associated with regulatory T cells are among the most enriched in the tumors that have immune infiltrates. This, in combination with the lack of PD-L1 on tumor cells, suggests that more intense immunotherapy aimed at elimination regulatory T cells may overcome the immune evasion in ACC. Since regulatory T cells expresses much higher levels of CTLA-4 than other immune cell-types, the inventors had the insight that one of the most efficient way to target regulatory T cells may be to use anti-CTLA-4 antibodies with the Fc portion capable antibody-dependent cell mediated cytotoxicity (ADCC) or phagocytosis (ADCP).

While the approved anti-CTLA-4 antibody Ipilimumab is considered ADCC/ADCP-competent due to its IgG1 Fc, its ADCC activity for Treg has been shown to less than optimal, primarily due to its down-regulation of CTLA-4 molecular through lysosomal degradation. Furthermore, since clinically used anti-CTLA-4 antibodies cause immunotherapy-related adverse events at high rate, the dose and duration of the treatment are severely curtailed in clinic. Preclinical and clinical studies have shown that due to its ability to avoid CTLA-4 down-regulation, anti-CTLA-4 antibodies such as ONC-392 have a much improved therapeutic index.

Described in more detail below, ONC-392 is a humanized monoclonal antibody generated by grafting the CDR regions from the mouse monoclonal antibody onto a human IgG1 antibody framework. To facilitate antibody-dependent cellular cytotoxicity (ADCC) for tumor rejection, ONC-392 has a human IgG1 Fc region, which has strong ADCC activity. Furthermore, to enhance ADCC activity, the Fc domain of ONC-392 is modified to improve the antibody's immunotherapeutic effects. Specifically, three mutations (S298A, E333A and K334A) are introduced in the CH to increase ADCC activity. An additional three mutations (M252Y, S254T and T256E) are introduced to increase the half-life of the antibody in vivo. The ONC-392 drug product is a sterile liquid for intravenous (IV) administration supplied in a 20 mL glass vial with flip-off seal over a 20 mm rubber stopper. The drug product is formulated in 20 mM histidine buffer, 8.8% (w/v) trehalose dihydrate, 0.06% (w/v) PS80, at pH 6.0 to a protein concentration of 5.0 mg/mL.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

### 2. Anti-CTLA-4 Antibody

Provided herein is an anti-CTLA-4 antibody. The anti-CTLA-4 antibody may be one or more of Ipilimumab/Yervoy, XTX101, Botensilimab, Zalifrelimab, ADG116, HBM4003, APL-509, BA3071, BMS-986249, TikAb, and quavonlimab (MK-1308). The anti-CTLA-4 antibody may also be described in U.S. Pat. No. 10,618,960, the contents of which are incorporated herein by reference.

The anti-CTLA-4 antibody may comprise a light chain variable region comprising a complementarity determining region (CDR) 1 comprising the amino acid sequence RASENIYSNLA (SEQ ID NO: 1); a CDR2 comprising the amino acid sequence AATNLQS (SEQ ID NO: 2) (LC1), AATNLQD (SEQ ID NO: 3) (LC2), or AATSLQS (SEQ ID NO: 4) (LC3); and, a CDR3 comprising the amino acid sequence QHLWGTPYT (SEQ ID NO: 5).

The light chain variable region comprising one of LC1-LC3 may also comprise one of the following sequences, respectively:

LC1
(SEQ ID NO: 11)
DIQMTQSPSSLSASVGDRVTITCRASENIYSNLAWYQQKPGKAPKLLLY
AATNLQSGVPSRFSGSGSGTDFTLTISSLQPEDFATYYCQHLWGTPYTF
GGGTKLEIK
LC2
(SEQ ID NO: 12)
DIQMTQSPSSLSASVGDRVTITCRASENIYSNLAWYQQKQGKAPKLLLY
AATNLQDGVPSRFSGSGSGTDYTLTISSLQPEDFATYFCQHLWGTPYTF
GQGTKLEIK
LC3
(SEQ ID NO: 13)
DIQMTQSPSSLSASVGDRVTITCRASENIYSNLAWYQQKPGKAPKLLIY
AATSLQSGVPSRFSGSGSGTDFTLTISSLQPEDFATYYCQHLWGTPYTF
GGGTKVEIK

More particularly, the light chain comprising one of LC1-LC3 may comprise one of the following amino acid sequences, respectively:

LC1
(SEQ ID NO: 22)
DIQMTQSPSSLSASVGDRVTITCRASENIYSNLAWYQQKPGKAPKLLLY
AATNLQSGVPSRFSGSGSGTDFTLTISSLQPEDFATYYCQHLWGTPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*
LC2
(SEQ ID NO: 23)
DIQMTQSPSSLSASVGDRVTITCRASENIYSNLAWYQQKQGKAPKLLLY
AATNLQDGVPSRFSGSGSGTDYTLTISSLQPEDFATYFCQHLWGTPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*
LC3
(SEQ ID NO: 24)
DIQMTQSPSSLSASVGDRVTITCRASENIYSNLAWYQQKPGKAPKLLIY
AATSLQSGVPSRFSGSGSGTDFTLTISSLQPEDFATYYCQHLWGTPYTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*

The anti-CTLA-4 antibody may comprise a heavy chain variable region comprising a CDR1 comprising the amino acid sequence GFSLTSYGLS (SEQ ID NO: 6); a CDR2 comprising the amino acid sequence YIWYDGNTNFHPSLKSR (SEQ ID NO: 7) (HC1), YIWYDGNTNFHSSLKSR (SEQ ID NO: 8) (HC2); or, YIWYDGNTNFHSPLKSR (SEQ ID NO: 9) (HC3); and, a CDR3 comprising the amino acid sequence TEGHYYGSNYGYYALDY (SEQ ID NO: 10).

The heavy chain variable regions comprising one of HC1-HC3 may comprise one of the following amino acid sequences, respectively:

HC1
(SEQ ID NO: 14)
QVQLQESGPGLVKPSETLSLTCTVSGFSLTSYGLSWIRQPPGKGLEWIG
YIWYDGNTNFHPSLKSRVTISKDTSKNQFSLKLSSVTAADTAVYYCAKT
EGHYYGSNYGYYALDYWGQGTSVTVSS
HC2
(SEQ ID NO: 15)
QVQLQESGPGLVKPSETLSLTCTVSGFSLTSYGLSWIRQPPGKGLEWIG
YIWYDGNTNFHSSLKSRVTISKDTSKSQVSLKLSSVTAADTAVYYCAKT
EGHYYGSNYGYYALDYWGQGTLVTVSS
HC3
(SEQ ID NO: 16)
QVQLQESGPGLVKPSETLSLTCTVSGFSLTSYGLSWIRQPPGKGLEWIG
YIWYDGNTNFHSPLKSRVTISVDTSKNQFSLKLSSVTAADTAVYYCAKT
EGHYYGSNYGYYALDYWGQGTLVTVSS

The anti-CTLA-4 antibody may comprise a heavy chain constant region from a human Ig protein, which may be IgG, IgE, IgM, IgD, IgA, IgY, IgG1, IgG2, IgG3, IgG4, IgA1 or IgA2. In one example, the constant region is a Fc region from a human IgG1 protein. In one example, the heavy chain constant region comprises the amino acid sequence:

(SEQ ID NO: 17)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPAPIEKTISKAKGQPREPQVYTLP
PSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG

The heavy chain constant region may also comprise one or more mutations. Relative to the sequence set forth in SEQ ID NO: 17, the one or more mutations may be selected from M135Y, S137T, T139E, S181A, E216A, and K217A, and a combination thereof. In one example, the heavy chain constant region of the antibody comprises all six mutations. The mutant heavy chain constant region may comprise the amino acid sequence:

(SEQ ID NO: 18)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPELLGGPSVFLFPPKPKDTLYITREPE
VTCVVVDVSHEDPEVKFNWYVDGVEVHNAKTKPREEQYNATYRVVSV
LTVLHQDWLNGKEYKCKVSNKALPAPIAATISKAKGQPREPQVYTLP
PSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG

Even more specifically, the heavy chain of the anti-CTLA-4 antibody comprising heavy chain variable regions HC1-HC3 may comprise one of the following amino acid sequences, respectively:

HC1
(SEQ ID NO: 19)
QVQLQESGPGLVKPSETLSLTCTVSGFSLTSYGLSWIRQPPGKGLEW
IGYIWYDGNTNFHPSLKSRVTISKDTSKNQFSLKLSSVTAADTAVYY
CAKTEGHYYGSNYGYYALDYWGQGTSVTVSSASTKGPSVFPLAPSSK
STSGGTAALGCLVKDYFPEPVTVSWNSGALTSGVHTFPAVLQSSGLY
SLSSVVTVPSSSLGTQTYICNVNHKPSNTKVDKKVEPKSCDKTHTCP
PCPAPELLGGPSVFLFPPKPKDTLYITREPEVTCVVVDVSHEDPEVK
FNWYVDGVEVHNAKTKPREEQYNATYRVVSVLTVLHQDWLNGKEYKC
KVSNKALPAPIAATISKAKGQPREPQVYTLPPSRDELTKNQVSLTCL
VKGFYPSDIAVEWESNGQPENNYKTTPPVLDSDGSFFLYSKLTVDKS
RWQQGNVFSCSVMHEALHNHYTQKSLSLSPG**
HC2
(SEQ ID NO: 20)
QVQLQESGPGLVKPSETLSLTCTVSGFSLTSYGLSWIRQPPGKGLEW
IGYIWYDGNTNFHSSLKSRVTISKDTSKSQVSLKLSSVTAADTAVYY
CAKTEGHYYGSNYGYYALDYWGQGTLVTVSSASTKGPSVFPLAPSSK
STSGGTAALGCLVKDYFPEPVTVSWNSGALTSGVHTFPAVLQSSGLY
SLSSVVTVPSSSLGTQTYICNVNHKPSNTKVDKKVEPKSCDKTHTCP
PCPAPELLGGPSVFLFPPKPKDTLYITREPEVTCVVVDVSHEDPEVK
FNWYVDGVEVHNAKTKPREEQYNATYRVVSVLTVLHQDWLNGKEYKC
KVSNKALPAPIAATISKAKGQPREPQVYTLPPSRDELTKNQVSLTCL
VKGFYPSDIAVEWESNGQPENNYKTTPPVLDSDGSFFLYSKLTVDKS
RWQQGNVFSCSVMHEALHNHYTQKSLSLSPG**
HC3
(SEQ ID NO: 21)
QVQLQESGPGLVKPSETLSLTCTVSGFSLTSYGLSWIRQPPGKGLEW
IGYIWYDGNTNFHSPLKSRVTISVDTSKNQFSLKLSSVTAADTAVYY
CAKTEGHYYGSNYGYYALDYWGQGTLVTVSSASTKGPSVFPLAPSSK
STSGGTAALGCLVKDYFPEPVTVSWNSGALTSGVHTFPAVLQSSGLY
SLSSVVTVPSSSLGTQTYICNVNHKPSNTKVDKKVEPKSCDKTHTCP
PCPAPELLGGPSVFLFPPKPKDTLYITREPEVTCVVVDVSHEDPEVK
FNWYVDGVEVHNAKTKPREEQYNATYRVVSVLTVLHQDWLNGKEYKC
KVSNKALPAPIAATISKAKGQPREPQVYTLPPSRDELTKNQVSLTCL
VKGFYPSDIAVEWESNGQPENNYKTTPPVLDSDGSFFLYSKLTVDKS
RWQQGNVFSCSVMHEALHNHYTQKSLSLSPG**

A C-terminal lysine (K) may be additionally included in the amino acid sequence of the heavy chains set forth in SEQ ID NOs: 19-21, which may increase expression levels. The terminal lysine may be cleaved naturally during production of the anti-CTLA-4 antibody, or upon administration of the antibody.

PP4637 (LC2/HC3): In one example the anti-CTLA-4 antibody comprises a light chain variable region comprising a CDR1 comprising the sequence set forth in SEQ ID NO: 1, a CDR2 comprising the sequence set forth in SEQ ID NO: 3, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 9, and a CDR3 comprising the sequence set forth in SEQ ID NO: 10. In particular, the light chain variable region may comprise the sequence set forth in SEQ ID NO: 12 and the heavy chain variable region may comprise the sequence set forth in SEQ ID NO: 16. More particularly, the light chain may comprise the sequence set forth in SEQ ID NO: 23, and the heavy chain may comprise the sequence set forth in SEQ ID NO: 21. This antibody may be referred to as ONC-392.

PP4631 (LC2/HC1): In another example, the anti-CTLA-4 antibody comprises a light chain variable region comprising a CDRI comprising the sequence set forth in SEQ ID NO: 1, a CDR2 comprising the sequence set forth in SEQ ID NO: 3, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 7, and a CDR3 comprising the sequence set forth in SEQ ID NO: 10. In particular, the light chain variable region may comprise the sequence set forth in SEQ ID NO: 13 and the heavy chain variable region may comprise the sequence set forth in SEQ ID NO: 14. More particularly, the light chain may comprise the sequence set forth in SEQ ID NO: 23, and the heavy chain may comprise the sequence set forth in SEQ ID NO: 19.

PP4638 (LC3/HC3): In a further example, the anti-CTLA-4 antibody comprises a light chain variable region comprising a CDR1 comprising the sequence set forth in SEQ ID NO: 1, a CDR2 comprising the sequence set forth in SEQ ID NO: 4, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 9, and a CDR3 comprising the sequence set forth in SEQ ID NO: 10. In particular, the light chain variable region may comprise the sequence set forth in SEQ ID NO: 12 and the heavy chain variable region may comprise the sequence set forth in SEQ ID NO: 16. More particularly, the light chain may comprise the sequence set forth in SEQ ID NO: 24, and the heavy chain may comprise the sequence set forth in SEQ ID NO: 21.

### 3. Dosing Regimens

The anti-CTLA-4 antibody may be administered to a subject, which may be a human. The administration may be to treat an ACC as described further herein. The anti-CTIA-4 antibody may be administered systemically, which may be via injection or intravenous (IV) administration. The antibody may be administered as a monotherapy, or as a combination therapy. The dosing regimen may comprise administering one or more doses of the anti-CTLA-4 antibody. Independently, each dose may be about 0.1 mg/kg, 0.3 mg/kg, 1 mg/kg, 3 mg/kg, 5, mg/kg, 6 mg/kg, 10 mg/kg, 15 mg/kg, 20 mg/kg, 25 mg/kg, 30 mg/kg, 50 mg/kg, or 100 mg/kg, or an amount in a range of two of these amounts. The dosing regimen may comprise periodic dosing, in which one of the foregoing doses is administered to the subject. At each cycle of dosing, the dose may be different from a previous dose. The dosing may involve escalating doses. In one example, the anti-CTLA-4 antibody is administered about every 1, 2, 3, 4, 5, or 6 weeks. In particular, the antibody is administered about every 3 weeks. When describing the period of a dosing cycle, “about” may mean±1, 2, or 3 days.

In particular, the dose of the anti-CTLA-4 antibody may be about 1, 3, 6, or 10 mg/kg, or an amount in a range of two of these amounts. The dosing regimen may also comprise 10 mg/kg for two doses, followed by 1-6 mg/kg of extended dosing (that is, each subsequent dose is 1-6 mg/kg). The extended dosing may comprise administering a dose of 3 mg/kg or 6 mg/kg. In one example, each administration is once about every 3 weeks. In one example, the anti-CTLA-4 antibody is administered once every about 4 weeks. The dosing may take place over a period of about 3, 6, 9, 12, 15, 18, 21, 24, 27, 30, 33, 36, 39, 42, 45, 48, 51, or 52 weeks, or within a range thereof.

The anti-CTLA-4 antibody may be administered in combination, either separately or mixed with, a second therapeutic agent. The therapeutic agent may be an anti-cancer agent. In one example, the anti-cancer agent is administered on the same day as the anti-CTLA-4 antibody. In particular, the anti-cancer agent may be an anti-PD-1 or anti-PD-L1 antibody. In a specific example, the anti-cancer agent is Pembrolizumab (KEYTRUDA). In one example, Pembrolizumab is administered at 200 mg/cycle, every 21 days. In a further example, the second therapeutic agent is administered on the same day as the anti-CTLA-4 antibody.

### 4. Formulations

The anti-CTLA-4 antibody may be formulated at or to provide a dose described herein. In one example, the formulation comprises 1, 2, 3, 4, 5, 6, 7, 8, 9, 10, 15, 20, 25, 30, 50, or 100 mg/mL of the anti-CTLA-4 antibody, or an amount in a range thereof. In one example, the amount is 5 mg/mL. The formulation may comprise 5, 10, 15, 20, 25, 30, 35, or 40 mM histidine buffer, or an amount in a range of two of these amounts. In one example, the amount is 20 mM. The formulation may also comprise 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% (w/v) α, α-trehalose dihydrate, or an amount in a range of two of these amounts. In one example, the amount is 8.8%. The formulation may comprise 0.01, 0.02, 0.03, 0.04, 0.05, 0.06, 0.07, 0.08, 0.09, or 0.10 (w/v) polysorbate 80, or an amount in a range of two of these amounts. In one example, the amount is 0.06%. The formulation may of a pH of 5.5, 5.6, 5.7, 5.8, 5.9, 6.0, 6.1, 6.2, 6.3, 6.4, or 6.5, or a pH in a range thereof. Equivalent ingredients to histidine buffer, a, a-trehalose dihydrate, and polysorbate 80 for formulating antibodies are known in the art, and may also be used as substitutes.

The formulation may comprise 5-100 mg/mL anti-CTLA-4 antibody, 20 mM histidine buffer, 8.8% (w/v) α, α-trehalose dihydrate, and 0.01-0.06% (w/v) polysorbate 80. The formulation may be at pH 6.0. In another example, the formulation comprises 5 mg/mL anti-CTLA-4 antibody, 20 mM histidine buffer, 8.8% (w/v) α, α-trehalose dihydrate, and 0.06% (w/v) polysorbate 80, at pH 6.0.

### 5. Cancer Treatment

The compositions and dosing regimens described herein may be used to treat cancer. The cancer may be a solid tumor. The cancer may be one of progressive locally advanced and metastatic cancer. The cancer may be stage IV cancer. The subject may exhibit failure or intolerance to standard of care guidelines, which may be National Comprehensive Cancer Network Clinical Practice Guidelines in Oncology (NCCN Guidelines). The cancer may be refractory or resistant to anti-PD-1/PD-L1 treatment. The resistance may be primary resistance or acquired resistance with disease progression after immunotherapy. The primary PD-1 resistance may be defined as disease progression within 24 weeks of initiation of anti-PD-(L)1 therapy. The acquired PD-1 resistance may be defined as 24 weeks or more of disease control (CR, PR or SD) after initiation of anti-PD-(L)1 therapy and has subsequently progressed after 24 weeks. The cancer may be immunotherapy naïve, and may be PD-L1 positive, such as by having PD-L1 Tumor Proportion Score ≥1%. The cancer may be non-small cell lung cancer. In another example, the cancer is ovarian, cervical, gastroesophageal, lung, or ovarian cancer.

The subject may have metastatic disease or locally advanced disease not amenable to local therapy. The subject may also have failed established standard medical anti-cancer therapies, which may be other than Pembrolizumab for a given tumor type, or may have been intolerant to such therapy.

The cancer may be a neoplasm or tumor resulting from abnormal uncontrolled growth of cells. The cancer may be a leukemia or lymphoma. The cancer may also involve cells that have the potential to metastasize to distal sites.

The cancer may be one of the following: a carcinoma, such as that of the bladder, breast, colon, kidney, liver, lung, ovary, pancreas, stomach, cervix, thyroid or skin; squamous cell carcinoma; a hematopoietic tumor of lymphoid lineage, such as a leukemia, acute lymphocytic leukemia, acute lymphoblastic leukemia, B-cell lymphoma, T-cell lymphoma, or Berkitt's lymphoma; a hematopoietic tumor of myeloid lineage, such as acute and chronic myelogenous leukemia or promyelocytic leukemia; a tumor of mesenchymal origin, such as fibrosarcoma or rhabdomyoscarcoma; a tumor such as melanoma, seminoma, tetratocarcinoma, neuroblastoma, or glioma; a tumor of the central and peripheral nervous system, such as astrocytoma, neuroblastoma, glioma, or schwannoma; a tumor of mesenchymal origin, such as fibrosarcoma, rhabdomyosarcoma, or osteosarcoma; or a tumor such melanoma, xenoderma pegmentosum, keratoactanthoma, seminoma, thyroid follicular cancer, or teratocarcinoma.

The cancer may be caused by aberrations in apoptosis. The cancer may be a follicular lymphomas, a carcinoma with one or more p53 mutations, a hormone-dependent tumor of the breast, prostate or ovary, a precancerous lesion such as familial adenomatous polyposis, or a myelodysplastic syndrome. The cancer may be a malignancy or dysproliferative change (such as metaplasia or dysplasia), or a hyperproliferative disorders, and may be in the ovary, bladder, breast, colon, lung, skin, pancreas, or uterus. In particular, the cancer may also be sarcoma, melanoma, or leukemia.

The cancer may also be non-small cell lung cancer, an advanced solid tumor, metastatic melanoma, metastatic head and neck carcinoma, metastatic renal cell carcinoma, metastatic colorectal cancer, sarcomas, metastatic prostate cancer, ovarian cancer, small cell lung cancer, metastatic breast cancer, pancreas cancer, gastric cancer, esophageal cancer, gastroesophageal junction adenocarcinoma, cervical cancer, adenoid cystic carcinoma (ACC), salivary gland cancer, or urothelial carcinoma.

In particular, the cancer may be ACC. The ACC may be recurrent, metastatic, or both. To treat ACC, the anti-CTLA-4 antibody may be administered once every about 3 or 4 weeks, particularly once every about 4 weeks. In one example, the anti-CTLA-4 antibody is the only immunotherapeutic agent administered to the subject. In another example, the anti-CTLA-4 antibody is administered as a monotherapy. In a further example, a second anti-cancer therapeutic is administered. The second anti-cancer therapeutic may be Pembrolizumab, which may be administered as described herein.

The present invention has multiple aspects, illustrated by the following non-limiting examples.

### Example 1

### Anti-CTLA-4 Antibody Treatment Safety and Efficacy

This example demonstrates safety and efficacy of the anti-CTLA-4 antibody ONC-392 (P4637) for treating cancer, particularly advanced solid tumors and non-small cell lung cancer (NSCLC). The trial was conducted in two parts: A and B. In Part A, patients were enrolled with a histologically or cytologically confirmed diagnosis of solid tumors who had progressive locally advanced or metastatic disease after failure of or intolerance to established standard medical anti-cancer therapies, as per standard of care guidelines, such as NCCN Guidelines.

In Part B, which evaluated dose finding and expansion cohorts, patients were enrolled with advanced/metastatic Non-Small Cell Lung Cancer (NSCLC) who were immunotherapy-naïve and PD-L1-positive (PD-L1 Tumor Proportion Score (TPS) ≥1%) or refractory/resistant to anti-PD-1/PD-L1 treatment (regardless of PD-L1 status).

### Summary of Study Design

A Phase IA/IB open label dose-escalation study of intravenous (IV) administration of ONC-392 drug product as a single agent and in combination with Pembrolizumab (anti-PD-1, marketed as KEYTRUDAR by Merck), was conducted in participants with advanced/metastatic solid tumors and NSCLC.

The study consisted of two linked parts:

Part A was a dose-finding rapid titration, Phase IA trial of ONC-392 as a single agent in patients with advanced disease of various histology. The aim of this trial was to define the recommended Phase II dose for ONC-392 monotherapy (RP2D-M).

Part B was a Phase IA/IB trial of ONC-392 in combination with a standard dose of 200 mg Pembrolizumab in patients with NSCLC. The trial consisted of a dose-finding, dose escalation or de-escalation, Phase IA component aimed at defining the recommended phase II dose for ONC-392 in combination with a standard dose of Pembrolizumab (RP2D-C), then progressing into two parallel, single arm, Phase IB expansion cohorts to test for safety and initial efficacy in two groups of patients with NSCLC:
Stage IV NSCLC anti-PD(L)1 immunotherapy naïve with PD-L1-positive (PD-L1 TPS≥1%);
Stage IV NSCLC refractory/resistant to anti-PD(L)1 immunotherapy regardless of PD-L1 status.

In the Phase IA component both anti-PD(L)1 immunotherapy naïve and refractory/resistant disease were enrolled.

### Trial Design and Sample Size

To be eligible for the study, patients had to be 18 years of age or older, had to have metastatic disease or locally advanced disease not amenable to local therapy, and had to have failed established standard medical anti-cancer therapies other than Pembrolizumab for a given tumor type, or have been intolerant to such therapy, or in the opinion of the Investigator have been considered ineligible for a particular form of standard therapy on medical grounds. Patients must have had an Eastern Cooperative Oncology Group (ECOG) performance status of ≤2.

A minimum of 10 patients and a maximum of 30 subjects were to be enrolled in Part A ONC-392 monotherapy.

Up to 18 subjects were to be enrolled in the Part B Phase IA ONC-392 and

Pembrolizumab combination therapy dose finding phase.

Two cohorts of 18 patients each were to be enrolled for Part B Phase IB ONC-392 and Pembrolizumab combination therapy dose finding phase.

Cohort 1: Eighteen (18) subjects were to be enrolled in the Part B Phase IB anti-PD (L)-1 naïve with PD-L1-positive (PD-L1 TPS ≥1%) cohort.

Cohort 2: Eighteen (18) subjects were be enrolled in the Part B Phase IB anti-PD (L)-1 refractory/resistant cohort.

In total, up to 84 subjects were to be enrolled in the entire study.

### Part A: ONC-392 Single Agent

The Part A Phase IA trial was tested up to five predefined dose levels: 0.1 mg/kg, 0.3 mg/kg, 1 mg/kg, 3 mg/kg and 10 mg/kg of ONC-392 as monotherapy through IV infusion every 21 days (Q3W). The trial used an accelerated titration design. Intra-patient dose escalation was tested in the first patient receiving 0.1 mg/kg, 0.3 mg/kg, 1.0 mg/kg without any AE. This patient was escalated to 3.0 mg/kg and received 3 cycles at this dose without any AE. The second patient started at 0.3 mg/kg without any AE. The enrollment was then converted to a 3+3 design at 3.0 mg/kg and 10.0 mg/kg levels in the protocol below.

### Part B: Combination of ONC-392 and Pembrolizumab in NSCLC

Part B was designed as a Phase IA dose escalation/de-escalation study followed by a Phase IB expansion component at the RP2D-C for the combination of ONC-392 with Pembrolizumab in two cohorts of patients with NSCLC.

The dose for Pembrolizumab was fixed at 200 mg/cycle dosed every 21 days (Q3W).

### Part B Phase IA Study

The Phase IA study was started at the dose one level below the RP2D-M dose for ONC-392 with 200 mg of Pembrolizumab and was to initially enroll 6 patients. The ONC-392 dose was to be adjusted according to following scenario:
(1) If 1/6 patients develop a dose limiting toxicity (DLT), then the dose one level below the RP2D-M were to be declared as RP2D-C.
Or:
(2) If 0/6 patients develop a DLT, 6 additional patients were to be enrolled at the RP2D-M dose level for ONC-392. If ≤1/6 of the additional patients developed a DLT, then the RP2D-M were to be declared as PR2D-C.
Or:
(3) When 2 DLTs occur before 6 patients were enrolled, the ONC-392 dose was to be de-escalated to the next dose level until ≤1/6 patients treated at that dose developed a DLT. This dose level was designated RP2D-C. If dose level 1 (0.1 mg/kg) was too toxic using the above rule, further exploration of the combination of the drugs was to be stopped.

### Part B Phase IB Study

The Part B Phase IB expansion cohorts were both designed for patients with advanced NSCLC and included an immunotherapy naïve cohort and a refractory/resistant cohort. The six patients treated at the RP2D-C in Part B Phase IA, were evaluable for efficacy. One of the aims of the expansion cohorts was to arrive at a more comprehensive safety profile for the combination of ONC-392 at the RP2D-C plus Pembrolizumab. To ensure the safety of the patients enrolled in the two expansion cohorts, a Pocock-type boundary was used to allow early stopping for excess toxicity at any given time. The trial was to be stopped at any point in time if the incidence of DLTs was significantly higher than θ=20%.

In the anti-PD(L)1 immunotherapy naïve population, patients with advanced NSCLC with PD-L1-positive (PD-L1 TPS ≥1% or otherwise indicated for Pembrolizumab) were to be included in the study. 18 subjects were to be enrolled for the Phase IB expansion cohort.

In the anti-PD(L)1 refractory/resistant population, patients with advanced NSCLC who had disease progression or did not tolerate anti-PD(L)1 containing treatment (including monotherapy or combination therapy, or immunotherapy combined with chemotherapy) after 4 or more cycles were to be included in this study. Prior CTLA-4 therapy was allowed. Prior history of irAE but recovered was allowed. 18 subjects were to be enrolled for the Phase IB expansion cohort.

The response rates for PD-(L)1 therapy naïve and refractory/resistant cohorts was to be determined separately at 6 months after the first treatment.

### Dosage/Dosage Form, Route, and Dose Regimen

For dose escalation in monotherapy, ONC-392 was administered as a minimal 30 minute IV infusion. Five dose levels of ONC-392 were to be evaluated: 0.1 mg/kg, 0.3 mg/kg, 1 mg/kg, 3 mg/kg and 10 mg/kg. The dosing interval was 21 days. ONC-392 was given at the schedule of Q3W. Intra-patient dose escalation up to 3 mg/kg were allowed. The treatment should continue for 4 additional cycles if the patient has confirmed complete remission, or confirmed disease progression if the patient tolerates the treatment. The treatment was to continue up to 1 year if the patient disease status was considered to be “stable disease” or “partial remission.” The treatment was to be stopped for unacceptable toxicity, or voluntary withdrawal from patient or 1 year, whichever occurred first.

In the combination of ONC-392 and Pembrolizumab, ONC-392 was to be administered first as a 30+15 min IV infusion. Pembrolizumab was to then be administered as a 30+15 minute IV infusion at a fixed 200 mg/dose. ONC-392 and Pembrolizumab were not be mixed in administration.

ONC-392 and Pembrolizumab were to be given at the schedule of Q3W. The treatment was to continue for 4 additional cycles if the patient had confirmed complete remission, or confirmed disease progression if the treatment can be tolerated. The treatment should continue up to 1 year if the patient disease status was considered to be “stable disease” or “partial remission.” The treatment was to be stopped for unacceptable toxicity, or voluntary withdrawal by the patient.

The administration of ONC-392, either as a single agent or in combination with Pembrolizumab, required the monitoring of vital signs every hour from the start of IV infusion to 4 hours after the end of infusion during dose escalation. Vital signs were to be monitored before and after infusion (+/−30 min) when patients were dosed at RP2D.

TABLE 1
Objectives and endpoints

| | | Objectives | Endpoints |
|---|---|---|---|
| Part A | Primary | Dose finding rapid titration | MTD or Recommended phase II dose for ONC-392 |
| | | | monotherapy (RP2D-M) |
| | Secondary | Characterize the PK profile | PK parameters |
| | | of single agent ONC-392 | |
| | Exploratory | Characterize anti-tumor | Objective response (PR, CR, SD or PD) as assessed |
| | | activity | by the Investigator based on RECIST1.1 over one |
| | | | year after patient receives first ONC-392 treatment. |
| Part B | Primary | Dose finding (Phase IA) | Recommended Phase II dose for ONC-392 in |
| | | | combination with a standard dose of |
| | | | pembrolizumab (RP2D-C) |
| | | Safety of RP2D-C (Phase IA | Incidence of Treatment Emergent Adverse Events |
| | | and IB) | (TEAEs) |
| | Secondary | Characterize the PK profile | PK parameters |
| | | of ONC-392 in combination | Objective response rate (ORR) as assessed by |
| | | with SOC Pembrolizumab | central reviewers based on RECIST1.1 at 6 months |
| | | Efficacy | and one year after patient receives first ONC-392 |
| | | | treatment. |
| | | | Progression-free survival (PFS) as assessed by the |
| | | | Investigator based on RECIST 1.1 and iRECIST. |
| | | | Overall survival (OS) following administration of |
| | | | ONC-392 in combination with Pembrolizumab. |

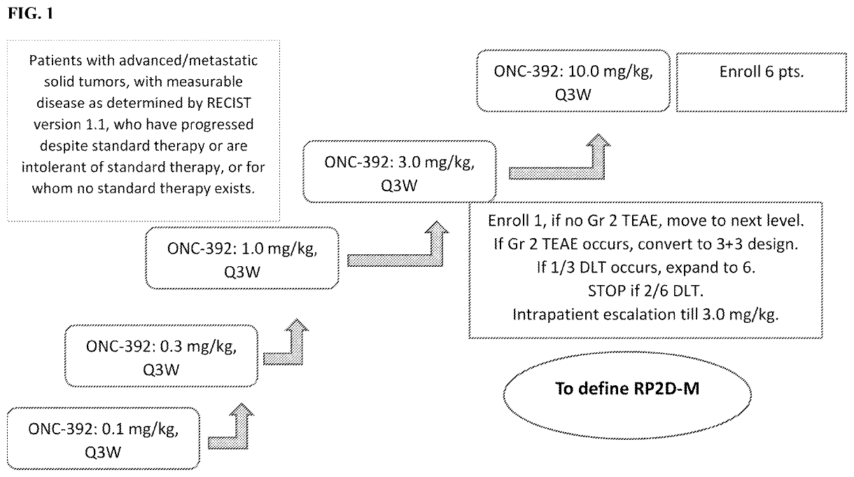

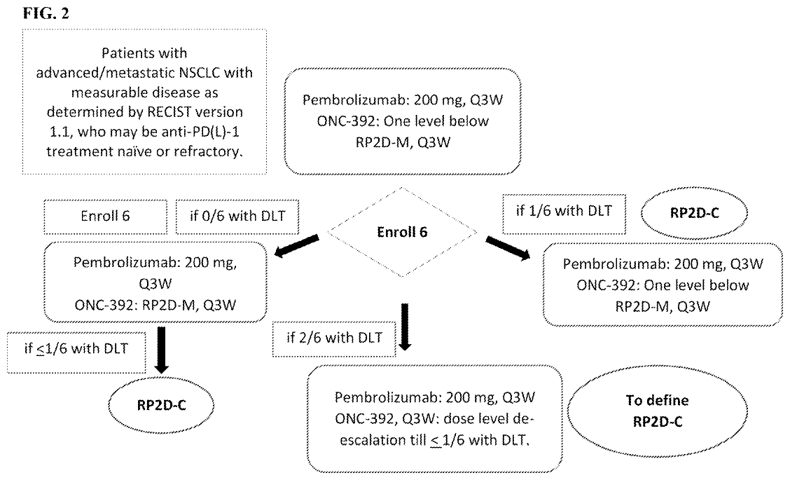

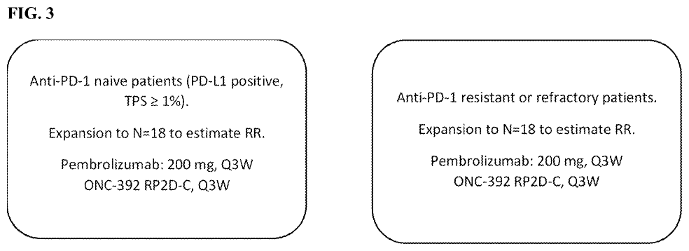

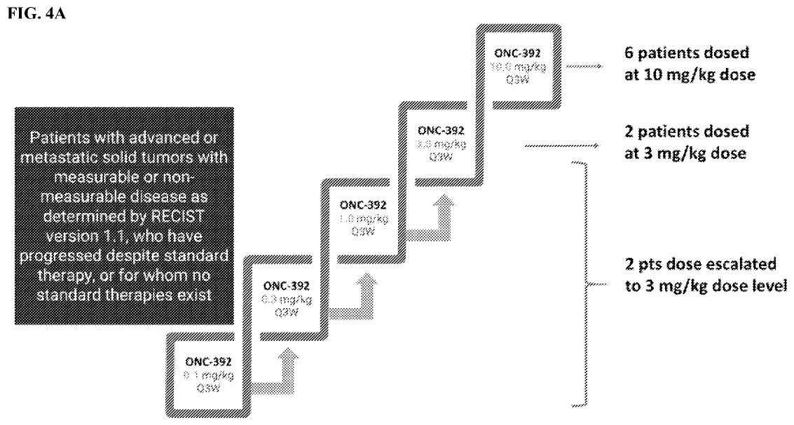

FIG. 1 shows a diagram of the ONC-392 monotherapy trial design. FIG. 2 shows a diagram of Part B of the phase I clinical trial of ONC-392 plus Pembrolizumab. FIG. 3 shows a diagram of Part B of the phase IB expansion of the trial of ONC-392 plus Pembrolizumab. The table below shows the study eligibility criteria.

TABLE 2
Study eligibility

| Inclusion Criteria | | Exclusion Criteria |
|---|---|---|
| 1. | Age ≥ 18 yr old. | 1 Patients who have not recovered to CTCAE ≤ 1 |
| 2. | Male or Female, Female must have | from the AE due to cancer therapeutics. The washout |
| | negative pregnancy test. | period for cancer therapeutic drugs (such as |
| 3. | Must have ≤ 2 ECOG scale. | chemotherapy, radioactive, or targeted therapy) should be |
| 4. | A histological or cytological | 5 half-lives of the drugs or 4 weeks for antibody drug. |
| | diagnosis of solid tumors and have | 2. Patients who are currently enrolled in a clinical |
| | progressive metastatic disease, or | trial of an investigational agent or device. |
| | progressive locally advanced disease | 3. Patients who are on chronic systemic steroid |
| | not amenable to local therapy (See | therapy at doses >10 mg/day |
| | detail in section 4.1) | 4. Patients who previously had a severe |
| 5. | Must have adequate organ function | hypersensitivity reaction to another mAb. |
| | as determined by laboratory tests | 5. Patients who have an active infection requiring |
| | (detail in section 4.1) | therapy and patients that have been given antibiotics |
| 6. | Patient has voluntarily agreed to | within 30 days of prior to administration of ONC-392 or |
| | participate by giving written | combined ONC-392 and Pembrolizumab. |
| | informed consent. | 6. Patients who have a history or current evidence of |
| 7. | Female patient agrees on | any condition, therapy, or laboratory abnormality that |
| | contraceptive methods. | might confound the results of the study, interfere with the |
| 8. | Male patient agrees on contraceptive | patient's participation for the full duration of the study, |
| | methods. | or is not in the best interest of the patient to participate, in |
| | | the opinion of the treating Investigator. |
| | | 7. Patients with known psychiatric or substance |
| | | abuse disorders that would interfere with cooperation |
| | | with the requirements of the trial. |
| | | 8. Patients who are pregnant, breastfeeding, or have |
| | | a positive pregnancy test result before the enrollment. |
| | | 9. For the Part B trial, the NSCLC patients that are |
| | | deemed to be not suitable for Pembrolizumab. |

### Safety Results of Part A

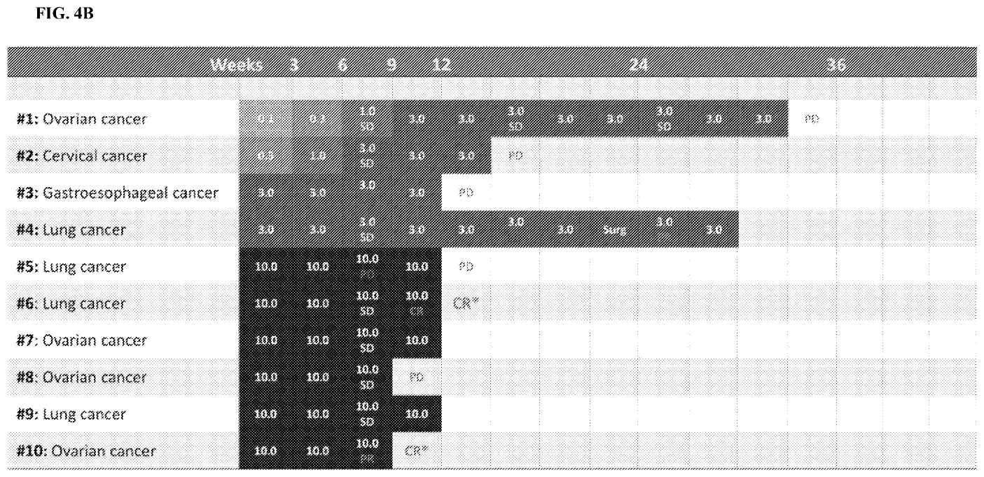

The dosing schedule for Part A of the clinical trial is shown in FIG. 4A. The demographics of the patients evaluated in Part A of the ONC-392 trial are shown in the following table.

TABLE 3

| Category | Number |
|---|---|
| Patients | 10 |
| Gender (F/M) | 7/3 |
| White/Asian/Black | 6/3/1 |
| Median age (range) | 62 (43-81) |

| Cancer type | n/stage |
|---|---|
| NSCLC | 4/IV |
| Ovarian cancer | 4/IV |
| GE junction cancer | 1/IV |
| Cervical cancer | 1/IV |

The dosing and preliminary outcomes of the trial are shown in FIG. 4B. Results indicated that ONC-392 at both doses was generally well-tolerated. Grade 3 irAEs of pancreatitis and colitis were manageable and reversible. PR2D for monotherapy was 10 mg/kg, q3w.

The results show that ONC-392 was well tolerated. The longest dosing was 3 mg/kg for up to 9 cycles. No DLT or Grade 3/4 AEs occurred during the DLT observation period at any dose. The maximum tolerable dose was not reached. The recommended phase 2 dose for monotherapy was determined to be 10 mg/kg. The following Grade 3/4 AEs occurred in three patients after 3 or 4 cycles of treatment at 10 mg/kg ONC-392: colitis/hypokalemia (2) and pancreatitis (1). Two of these three patients had unconfirmed complete response, and one had stable disease with shrinking tumor burden. Other drug-related AEs were grade 1/2, and those that occurred in more than two patients included infusion-related reactions, pruritis, fatigue, and TSH increase.

### Clinical Results

In addition, beneficial activity was observed in 6/10 patients. Two of 6 patients treated at 10 mg/kg ONC-392 exhibited complete response, two of 6 patients treated at 10 mg/kg ONC-392 had stable disease with a significant reduction of tumor burden or a biomarker of enhanced T cell activation in the tumor, and two out of 4 patients treated at 3 mg/kg had stable disease (SD) at greater than 7 months. Stable disease was observed in 7 out of 10 patients, and partial response was observed in 1 out of 10 patients in the first tumor assessment. Further, clinical improvements were observed among three PD-(L)1 refractory/resistant patients with NSCLC (one with complete response; one with disease control at greater than 24 weeks who became eligible for surgery; and, one with stable disease at 8 weeks with continued treatment).

### Safety and Efficacy Conclusion

ONC-392 was generally safe and well tolerated. Treatment-related AEs could be managed. And the maximum tolerable dose was not reached at the 10 mg/kg dose. ONC-392 also demonstrated therapeutic anti-tumor activities. As the first pH-sensitive monoclonal antibody that preserves CTLA-4 recycling and avoids lysosomal degradation, ONC-392 may fundamentally change the risk/benefit ratio of CTLA-4 targeting by conferring improved efficacy and reduced toxicity.

### Example 2

### Clinical Trial on the Efficacy of an Anti-CTLA-4 Antibody Against ACC

### Summary of Study Design

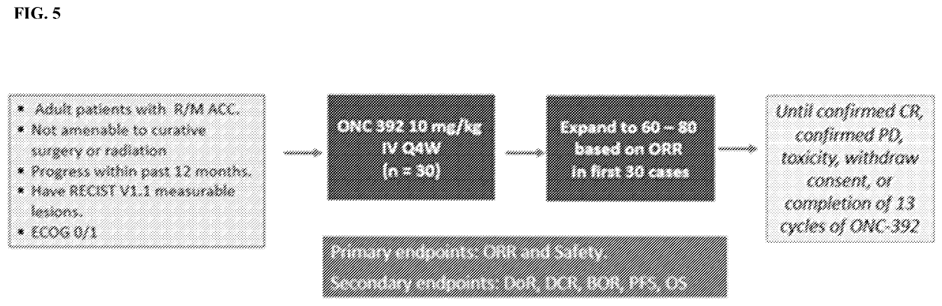

The study was part of a Phase IA/IB/II, open label, dose-escalation, and dose-expansion study of intravenous (IV) ONC 392 as a single agent and in combination with Pembrolizumab (anti PD-1, marketed as KEYTRUDA® by Merck) in patients with advanced/metastatic solid tumors. Part D aimed at studying ACC patients treated with ONC-392 monotherapy. A schematic of the study design is shown in FIG. 5.

The patients had recurrent and/or metastatic adenoid cystic carcinoma (R/M ACC) that originated from the salivary gland or from other locations and was not amenable to curative intent surgery or radiation. Prior chemotherapy, targeted therapy, and immunotherapy were allowed. The primary endpoint was objective response rate (ORR) as assessed by blinded independent central review (BICR) based on Response Evaluation Criteria in Solid Tumors version 1.1 (RECIST 1.1) after the patient received the first ONC-392 treatment either as monotherapy or as combination therapy with pembrolizumab. The primary safety endpoint was incidence of Treatment Emergent Adverse Events (TEAEs). Secondary efficacy parameters included objective response rate (ORR), duration of response (DoR), best overall response (BoR), and disease control rate (DCR). Additional efficacy parameters included Progression-free survival (PFS) as assessed by the Investigator based on RECIST 1.1 and iRECIST; overall survival (OS) following administration of ONC-392; and ORR, DoR, BoR, DCR as assessed by the BICR based on iRECIST. In addition, PK parameters were measured to investigate any exposure-response correlation.

### Dosage/Dosage Form, Route, and Dose Regimen

For dose escalation in monotherapy, five dose levels of ONC-392 were evaluated: 0.1 mg/kg, 0.3 mg/kg, 1.0 mg/kg, 3.0 mg/kg and 10 mg/kg. ONC-392 was administered as an IV infusion over a minimum of 30 minutes for dose levels of 0.1, 0.3, and 1.0 mg/kg and a minimum of 60 minutes for the 3.0 mg/kg dose level. At the 10 mg/kg dose level, a minimum of 90 minutes of infusion time was required for the first dose, and a minimum of 60 minutes for subsequent doses. The ONC-392 dosing interval was 21 days (every 3 weeks [Q3W]). Intrapatient dose escalation up to 3 mg/kg was allowed.

For the combination of ONC-392 and Pembrolizumab, ONC-392 was administered first as an IV infusion over a minimum of 60 minutes except that the first dose of ONC-392 10 mg/kg was administrated over a minimum of 90 minutes. For the 6.0 mg/kg ONC-392 dose level, the IV infusion was given over 60 minutes. Pembrolizumab was then be administered IV over a minimum of 30 minutes at a fixed 200 mg/dose. There was a gap of at least 30 minutes between the end of the ONC-392 infusion and the start of the Pembrolizumab infusion. ONC-392 and Pembrolizumab was not mixed during administration. ONC-392 and Pembrolizumab were both given Q3W.

Study treatment (both monotherapy and combination therapy) could be continued for 4 additional cycles (optional) after a patient had confirmed progressive disease (PD) based on immune Response Evaluation Criteria in Solid Tumors (iRECIST) if the patient tolerated the treatment.

Study treatment (both monotherapy and combination therapy) was stopped for unacceptable toxicity, voluntary withdrawal by the patient, or at 1 year (13- or 17 cycles), whichever occurs first (refer to Section 5.7 for options after 1 year).

The dosing for Part D of the study (on ACC) is ONC-392, 10 mg/kg, IV infusion, Q4W. The treatment period is up to 13 cycles (approximately 1 year). Dose adjustment was permitted.

Patient Selection, Enrollment, Discontinuation, and Withdrawal

### Patient Inclusion Criteria

Patients were at least 18 years of age on the day of signing informed consent. Patients could be male, or female if they tested negative on a pregnancy test. Patients must have had a performance status of ≤1 on the ECOG Performance Scale. Patients must have had a histological or cytological diagnosis of solid tumors and had progressive metastatic disease or progressive locally advanced disease.

For Part D, the patient inclusion criteria are:
a. Histological or cytologically confirmed adenoid cystic carcinoma (ACC) that had locally recurred or metastasized, not amenable to curative intent surgery or radiation. ACC arising from either salivary or non-salivary gland primary sites was allowed.
b. With measurable target lesion as determined by RECIST 1.1.
c. New or progressive lesion on radiologic imaging study performed within 12 months prior to study enrollment (progression of disease over any interval is allowed) and/or new/worsening disease related symptoms within 12 months prior to study enrollment. Note: This assessment was performed by the treating investigator. Photo with ruler reference or radiologic images to document the disease progression was required.
d. Prior chemotherapy or target therapy or immunotherapy was allowed.
e. Life expectancy ≥12 weeks.

Measurable disease as determined by RECIST 1.1 was determined as follows:
a. Tumor mass: Must be accurately measurable in at least 1 dimension (longest diameter to be recorded) with a minimum size of:
1. 10 mm by computed tomography (CT) scan (CT scan slide thickness must be <5 mm),
Or:
2. 20 mm by chest X-ray (if clearly defined and surrounded by aerated lung).
b. With or without malignant lymph nodes: ≥15 mm in short axis when assessed by CT scan (CT scan slice thickness must be <5 mm). The measurement was two dimensions at axial plane. The short axis was perpendicular to long diameter.

Patients must also have had adequate organ function as indicated by particular laboratory values. Patients voluntarily agreed to participate by giving written informed consent. If they had childbearing potential (WOCBP) and were sexually active, female patients agreed to use adequate and effective birth control starting with the first dose of study drug through 90 days after the last dose of study therapy. Male patients, if sexually active, agreed to use adequate and effective methods of contraception starting with the first dose of study drug through 90 days after the last dose of study therapy. Patients agreed to grant the study team access to archival diagnostic tissue (recut slides or tumor biopsy).

Patients were excluded based on the following criteria:
1) Patients who had not recovered to NCI CTCAE Grade 1 or better from AEs due to cancer therapeutics except chemotherapy associated peripheral neuropathy (motor or sensory), or endocrine related AE, in which recovery to ≤Grade 2 was allowed. The washout period for cancer therapeutic drugs (such as chemotherapy, radiation, or targeted therapy) was 21 days. The washout period for treatment regimen containing monoclonal antibodies was 28 days. Best supportive care, such as thyroxine, insulin, steroid replacement treatment, blood transfusion and therapy for non-cancer conditions were allowed.
2) Patients who were currently enrolled in any other clinical trial testing an investigational agent or using an investigational device, or concurrently in other approved systemic therapy.
3) Patients who were on chronic systemic steroid therapy at doses >10 mg/day prednisone or equivalent, or on any other form of immunosuppressive medication, within 7 days prior to ONC-392 treatment. Topical steroid use was allowed.
4) Active brain metastases or leptomeningeal metastases. Patients were eligible if brain metastases were adequately treated and patients were neurologically stable (except for residual signs or symptoms related to the central nervous system (CNS) treatment).
5) Patients who had an active infection requiring systemic IV antibiotics within 14 days prior to administration of ONC-392 or combined ONC 392 and Pembrolizumab. Regular treatment of urinary tract infection (UTI) and/or topical treatment were allowed.
6) Patients who had a history or current evidence of any condition, therapy, or laboratory abnormality that might confound the results of the study, interfere with the patient's participation for the full duration of the study, or make study participation not in the best interest of the patient.
7) Patients with known psychiatric or substance abuse disorders that would interfere with cooperation with the requirements of the trial.
8) Patients who were pregnant or breastfeeding ONC-392 IV Infusion

All patients had pre-medication in first treatment of ONC-392 to prevent infusion reaction. The pre-medications was recorded in concomitant medications.

ONC-392 IV infusion was given over a minimum of 30 min for dose levels of 0.1, 0.3, and 1.0 mg/kg and a minimum of 60 min for dose levels of 3.0 mg/kg and 6.0 mg/kg, a minimum of 90 minutes for the first dose 10.0 mg/kg, and a minimum of 60 minutes for subsequent dose of 10 mg/kg. For combination therapy, ONC-392 was given first and there was an interval of at least 30 min before the 200 mg fixed dose of IV Pembrolizumab was given over a minimum of 30 minutes.

The drug product of ONC-392 had a concentration of 5 mg/mL. The drug product was diluted with a 5% Dextrose Solution to a final concentration between 1.0 to 2.5 mg/mL. The IV infusion line had a 0.2 μm inline filter. After the infusion, a 25 mL normal saline solution was used to flush the line according to local institutional guidelines.

### Composition of the Drug Product

The ONC-392 drug product was a sterile liquid for IV administration supplied in a 20 ml glass vial with flip-off seal over a 20 mm rubber stopper. The drug product was formulated in 20 mM histidine buffer, 8.8% (w/v) trehalose dihydrate, 0.06% (w/v) PS80, at pH 6.0 to a protein concentration of 5.0 mg/mL. The target fill volume was 17.340 mL per vial for an extractable volume of 16 mL (80 mg). The long-term storage condition was 2° C. to 8° C., protected from light.

### Example 3

### Anti-CTLA-4 Antibodies are Effective Against ACC

This example demonstrates the efficacy of anti-CTLA-4 antibodies, including ONC-392, for treating ACC. ONC-392 generated a clinical response in an ACC patient, who was a 48 year-old female with the following clinical diagnosis and history: ACC in the left nasal cavity in January 2018. Posterior maxillary mass, biopsy on Jan. 29, 2018. Pathology diagnosis: ACC, Grade 2, cribriform pattern, involving bone. Immunohistochemistry testing indicated that the cells were positive for AE1/AE2; and negative for P63, TTF1, ER, PR, GATA3, GCDFP15, and CDX2. The cells showed strong positive staining for MYB and CAM5.2, and were negative for p16 and p40.

The patient received proton/neutron radiation at University of Washington in 2019. The tumor recurred in the left maxilla, and the patient underwent salvage surgery comprising a left subtotal maxillectomy/fibula free flap reconstruction/orbital floor reconstruction for recurrence in January 2020. The cancer recurred in March 2021 and the patient underwent a left revision total maxillectomy, left orbital exenteration, resection of malignant tumor from the anterior skull base and infratemporal fossa-extradural, left neck exploration with lysis of scar and preparation of vessels followed by left radial forearm free flap harvest with skin paddle, left thigh split thickness skin graft, and reconstruction of midface and orbital defect with free soft tissue transfer. Anterior skull base durotomy with spinal cord fluid leak was observed on Apr. 23, 2021. The patient received chemoradiation therapy between Jun. 8, 2021 and Jul. 22, 2021 with concurrent cisplatin therapy. The cancer grew progressively during the treatment, and lung metastasis with multiple bilateral nodules was diagnosed December 2021.

After enrolling in an ONC-392 clinical trial described in Example 2, the patient was assigned to the arm with 10.0 mg/kg of ONC-392, Q4W, using the ONC-392 drug product. The screening MRI (12/09/2021) on orbit, face and neck showed significantly increased bulk of left hemifacial mass. The lesion was centered in the orbit, supraorbital region and extended to the frontal sinuses, residual left maxillary sinus, and anterior left temporal soft tissues. It measured approximately 7.7×4.1 cm in largest transverse and AP dimensions. The patient had a first cycle of treatment of 10 mg/kg ONC-392 on Jan. 6, 2022 and a second cycle of treatment on Feb. 3, 2022.

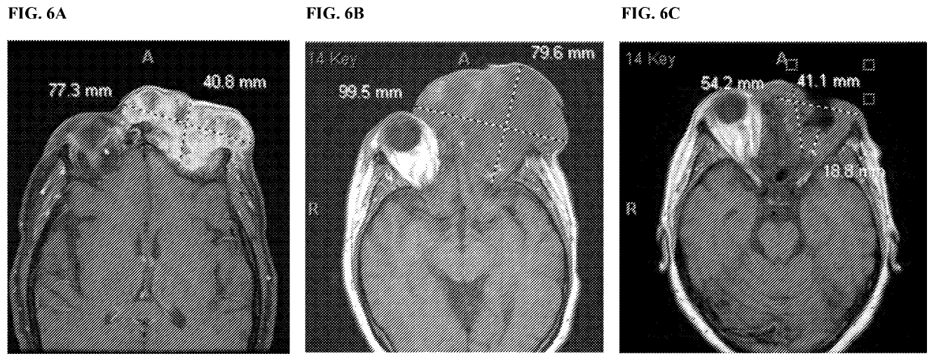

FIG. 6A shows the target lesion in an MRI scan taken of the patient on Dec. 9, 2021. The scan shows a tumor mass on left face and forehead of the ACC patient. After two doses of ONC-392, the patient showed apparent aggressive tumor growth, with a massive tumor bleeding event occurring two days before the 8-week MRI assessment from Feb. 24, 2022 shown in FIG. 6B. It was reported that the target lesion was an enlarging, expansile mass centered in the area of the left orbit measuring 10.0×7.4×8.9 cm, markedly increased in size compared to before. The mass appeared to invade the frontal sinuses as well as the left nasal cavity into the left sphenoid sinus. This mass also crossed midline anteriorly towards the right medial canthus soft tissues, progressed compared to before. There was no evidence of invasion into the right intraconal space. There was invasion of the left frontal calvarium through inner table, and lesion appeared to abut the left frontal lobe. A chest CT also showed interval increase in non-target lesions in the lung metastasis.

There was no other treatment option for the patient and the patient insisted on continuing the ONC-392 treatment. After a third dose of ONC-392 on Mar. 3, 2022, the facial tumor mass show rapid regression and had largely resolved within three weeks. An MRI scan of the lesion from Mar. 24, 2022 is shown in FIG. 6C. Since the previous exam from Feb. 24, 2022, there was a significant interval reduction in size of the expansile mass centered in the area of the left orbit, which corresponded to the biopsy confirming recurrent malignancy. The overall size was difficult to measure due to involvement of many surrounding structures and diffuse appearance of the mass. Grossly, the reduction in volume the mass was estimated to be about at least 75%. There was no radiographically pathologic adenopathy. Based on MRI evaluation, the main lesion in the head showed a 78% reduction of tumor burden from baseline. A chest CT showed the interval reduction of the metastatic lung lesions. The overall assessment on Mar. 24, 2022 indicated a partial response (PR) and the patient continued the treatment. The results from this patient indicate that anti-CTLA-4 antibodies can be used a sole immunotherapeutic agents to treat cancers including ACC.

What is claimed is:

1. A method of treating an adenoid cystic carcinoma (ACC) in a subject in need thereof, comprising administering an anti-CTLA-4 antibody to the subject.

2. The method of claim 1, wherein the ACC is recurrent, metastatic, or both.

3. The method of claim 1 or 2, wherein the anti-CTLA-4 antibody is the only immunotherapeutic agent administered to the subject.

4. The method of claim 3, wherein the anti-CTLA-4 antibody is administered as a monotherapy.

5. The method of any one of claims 1-4, wherein the anti-CTLA-4 antibody is selected from the group consisting of Ipilimumab/Yervoy, XTX101, Botensilimab, Zalifrelimab, ADG116, HBM4003, APL-509, BA3071, BMS-986249, TikAb, and quavonlimab (MK-1308), and a combination thereof.

6. The method of any one of claims 1-4, wherein the anti-CTLA-4 antibody comprises:
(a) a light chain variable region comprising a complementarity determining region (CDR) 1 comprising the amino acid sequence set forth in SEQ ID NO: 1; a CDR2 comprising the amino acid sequence set forth in any one of SEQ ID NOs: 2-4; and, a CDR3 comprising the amino acid sequence set forth in SEQ ID NO: 5; and,
(b) a heavy chain variable region comprising a CDRI comprising the amino acid sequence set forth in SEQ ID NO: 6; a CDR2 comprising the amino acid sequence set forth in any one of SEQ ID NOs: 7-9; and, a CDR3 comprising the amino acid sequence set forth in SEQ ID NO: 10.

7. The method of claim 6, wherein the anti-CTLA-4 antibody comprises a heavy chain comprising a Fc region of a human Ig protein.

8. The method of claim 7, wherein the human Ig protein is human IgG1.

9. The method of claim 8, wherein the Fc region of the human IgG1 protein comprises the sequence set forth in SEQ ID NO: 17 or 18.

10. The method of claim 6, wherein the anti-CTLA-4 antibody comprises a light chain variable region comprising a CDR2 comprising the sequence set forth in SEQ ID NO: 3 and heavy chain variable region comprising a CDR2 comprising the sequence set forth in SEQ ID NO: 9.

11. The method of claim 10, wherein the anti-CTLA-4 antibody comprises a light chain variable region comprising the sequence set forth in SEQ ID NO: 12 and a heavy chain variable region comprising the sequence set forth in SEQ ID NO: 16.

12. The method of claim 11, wherein the anti-CTLA-4 antibody comprises a light chain comprising the sequence set forth in SEQ ID NO: 23 and a heavy chain comprising the sequence set forth in SEQ ID NO: 21.

13. A method of treating an adenoid cystic carcinoma in a subject in need thereof, comprising administering an anti-CTLA-4 antibody to the subject, wherein the anti-CTLA-4 antibody comprises a light chain comprising the sequence set forth in SEQ ID NO: 23 and a heavy chain comprising the sequence set forth in SEQ ID NO: 21.

14. The method of claim 13, wherein the anti-CTLA-4 antibody has been diluted with 5% Dextrose Solution to a final concentration of about 1.0 to about 2.5 mg/mL, from a formulation containing 5 mg/mL anti-CTLA-4 antibody, 20 mM histidine buffer, 8.8% (w/v) trehalose dihydrate, and 0.06% (w/v) PS80 at pH 6.0.

15. The method of claim 13 or 14, wherein the anti-CTLA-4 antibody is the only immunotherapeutic agent administered to the subject.

16. The method of claim 15, wherein the anti-CTLA-4 antibody is administered as a monotherapy.

17. A composition comprising an anti-CTLA-4 antibody comprising:
(a) a light chain variable region comprising a complementarity determining region (CDR) 1 comprising the amino acid sequence set forth in SEQ ID NO: 1; a CDR2 comprising the amino acid sequence set forth in SEQ ID NO: 3; and, a CDR3 comprising the amino acid sequence set forth in SEQ ID NO: 5; and,
(b) a heavy chain variable region comprising a CDR1 comprising the amino acid sequence set forth in SEQ ID NO: 6; a CDR2 comprising the amino acid sequence set forth in SEQ ID NO: 9; and, a CDR3 comprising the amino acid sequence set forth in SEQ ID NO: 10,
wherein the anti-CTLA-4 antibody has been diluted with 5% Dextrose Solution to a final concentration of about 1.0 to about 2.5 mg/mL, from a formulation containing 5 mg/mL anti-CTLA-4 antibody, 20 mM histidine buffer, 8.8% (w/v) trehalose dihydrate, and 0.06% (w/v) PS80 at pH 6.0.

18. The composition of claim 17, wherein the light chain variable region comprises the sequence set forth in SEQ ID NO: 12 and the heavy chain variable region comprises the sequence set forth in SEQ ID NO: 16.
